(19) United States
(12) Patent Application Publication
(10) Pub. No.: US 2011/0230854 A1
(43) Pub. Date: Sep. 22, 2011

(54) TAMPON WITH A PERFORATED OUTER COVER

(75) Inventor: Steven Smet, Zele (BE)

(73) Assignee: ONTEX HYGIENEARTIKEL DEUTSCHLAND GMBH, Grosspostwitz (DE)

(21) Appl. No.: 13/129,104

(22) PCT Filed: Nov. 13, 2009
(86) PCT No.: PCT/EP2009/065089

§ 371 Date: May 12, 2011

(30) Foreign Application Priority Data
Nov. 13, 2008 (EP) 08169027.3

(51) Int. Cl. A61F 13/20

(52) U.S. Cl. 604/385.17

(57) ABSTRACT

A tampon is described which includes an elongated absorbent core and an outer cover. The tampon can include an elongated absorbent core, an upper insertion end, a lower withdrawal end, and an outer cover. The outer cover has a multiplicity of perforations. The degree of perforation in an upper cylindrical segment of said outer cover is larger than the degree of perforation of a lower cylindrical segment of said outer cover. The upper cylindrical segment is located in the upper part of said outer cover.

### FIELD OF THE INVENTION

The present invention relates to tampons, in particular tampons for feminine hygiene, including an elongated absorbent core and a perforated outer cover covering the absorbent core. The absorbent core has an upper insertion end, a lower withdrawal end, and a longitudinal main portion there between.

### BACKGROUND OF THE INVENTION

The use of tampons often results in leakage even at normal levels of menstrual fluid. This is due to absorption problem. Instead of passing through the tampon cover and being stored at its absorbent core, non-absorbed fluid will run over the outer surface of the tampon towards its withdrawal end. This results in leakage causing much discomfort to the wearer.

Many product designs have been proposed to increase the absorption across the outer surface and to reduce leakage. Such designs include different groove-and-rib structure systems or various cover material compositions and/or cover surface treatments and the like.

The absorption capacity of most conventional tampons is more than sufficient to handle the first amounts of liquid. However, solid state particles of the menstrual flow may be accumulated on the surface of the tampon. They fill and cover the pores of the outer cover of the tampon and obstruct the flow of liquid. This results in leakage at the withdrawal end.

U.S. Pat. No. 6,860,874 discloses, for example, tampons with an apertured outer cover wherein the size of the apertures increases towards the withdrawal end of the tampon. However, the small apertures provided in the upper part of such tampons may often be blocked by solid state particles. Thus, the entire vaginal fluid has to be absorbed in the lower part of the tampons, reducing the absorption capacity of the tampon.

Therefore, there exists a need to further improve the absorption of tampons having a perforated outer cover. More in particular, there exists the need to improve their performance especially in the frequent situations when vaginal fluid cannot be fully absorbed in time to avoid leakage.

### SUMMARY OF THE INVENTION

The aspects of the present invention address at least some, e.g., one or more, of the above discussed needs of the art.

In particular, the inventors realised that providing a tampon having an outer cover with specific perforations distribution and characteristics along the tampon, can greatly improve the absorption capacity of such tampons. By improving the permeability of the cover, the overall absorption capacity increases by increasing the practical absorption speed and thus the capacity. These characteristics allow an efficient use of the absorbent capacity of the tampon and avoid unpleasant leakage.

Accordingly, in an aspect, the invention provides a tampon comprising an elongated absorbent core, an upper insertion end, a lower withdrawal end, and an outer cover having a multiplicity of perforations, wherein the degree of perforation in an upper cylindrical segment is larger than the degree of perforation of a lower cylindrical segment in said outer cover and wherein said upper cylindrical segment is located in the upper part of said outer cover. The perforations may vary over the length of the tampon so that a differentiated expansion of the tampon while absorbing fluid is achievable.

The outer cover may provide smooth and non-drying feeling. Preferably, the outer cover may reduce resistance or friction upon insertion or removal of the tampon from a vaginal cavity. The outer cover may provide a clean appearance of the product after use. Such combinations of esthetical advantage and comfortable feeling are highly appreciated by users.

It has now been realised that by suitably choosing or distributing the degree of perforation along the length of the outer cover, the absorptive properties of the tampon may be improved, while simultaneously ensuring the non-drying feeling and comfort endowed by the outer cover.

Hence, where the outer cover defines a cylindrical segment comparably proximal to the insertion end of the tampon (e.g., an upper cylindrical segment as intended herein) having a comparably greater degree of perforation, this segment can enhance the absorptive properties of the tampon such as particularly by reducing the propensity of perforations or apertures of the outer cover to become blocked or obstructed by particulate matter or dense fluids. Where the outer cover defines a cylindrical segment comparably proximal to the withdrawal end of the tampon (e.g., a lower cylindrical segment as intended herein) having a comparably smaller degree of perforation, this segment can enhance the non-drying feeling, comfort and aesthetic quality of the tampon such as by preventing or reducing contact between the absorbent elements of the tampon and a user's body and/or by restraining the width-wise expansion of the tampon upon use.

Consequently, a tampon cover defining an upper cylindrical segment having a comparably greater degree of perforation than a lower cylindrical segment of the tampon cover provides for improved absorptive properties as well as an agreeable non-drying, pleasant feeling.

To pronounce these advantages to a particularly desirable extent preferably perceptible by users, the upper and lower cylindrical segments may span relatively substantial or sizeable portions of the outer cover of the tampon. Thus, a particular embodiment relates to a tampon comprising an elongated absorbent core, an upper insertion end, a lower withdrawal end, and an outer cover having a multiplicity of perforations, wherein the degree of perforation in an upper cylindrical segment is larger than the degree of perforation in a lower cylindrical segment in said outer cover and wherein said upper cylindrical segment is located in the upper part of said outer cover, and wherein the height (i.e., dimension in the longitudinal direction of the tampon) of said upper cylindrical segment is at least 20% of the height of the outer cover and the height of the lower cylindrical segment is at least 20% of the height of the outer cover.

Preferably, as also apparent from the drawings, cylindrical segments of the outer cover as intended herein, such as the upper and lower cylindrical segments as intended herein may be non-overlapping, i.e., may either adjoin one another or be spaced away from one another on the outer cover in the longitudinal direction of the tampon.

The preferred embodiments of the invention are described in the following sections and in the appended claims.

### SHORT DESCRIPTION OF THE FIGURES

FIG. 1 represents a tampon comprising an elongated absorbent core, an upper insertion end, a lower withdrawal end, and an outer cover according to an embodiment of the invention.

FIG. 2 represents a graph of the average perforation size distribution along the Z-axis of a tampon according to an embodiment of the invention.

FIG. 3 represents two segments of the outer cover located near the upper insertion end and near the lower withdrawal end of the tampon.

FIG. 4 represents the absorption over time of a tampon according to an embodiment of the invention and of a tampon having regular size perforations along its length.

FIG. 5 represents a schematic view of a tampon according to an embodiment of the invention wherein the outer cover comprises a multiplicity of perforations of different shape and size.

FIG. 6 represents a schematic view of a tampon according to an embodiment of the invention wherein the upper cylindrical segment comprises a combination of small and large perforations.

FIG. 7 represents a schematic view of a tampon according to an embodiment of the invention wherein the perforations are randomly distributed within a cylindrical segment.

FIG. 8 represents a schematic view of a tampon according to an embodiment of the invention wherein the outer cover has small perforations proximate to the insertion end and proximate to the withdrawal end.

FIG. 9 and FIG. 9bis represent a schematic view of a tampon according to an embodiment of the invention wherein the outer cover is provided with regularly shaped and homogeneously distributed perforations.

FIG. 10 represents a schematic view of a tampon provided with grooves on its outer surface and wherein the outer cover is provided with regularly shaped perforations and homogeneously distributed perforations according to an embodiment of the invention.

FIG. 11 represents a schematic view of a tampon after use wherein the upper surfaces of said upper and lower cylindrical segments have enlarged width compared to the lower surfaces of said upper and lower cylindrical segments.

FIG. 12 represents a schematic view of a tampon after use wherein the outer cover has small perforations proximate to the insertion end and proximate to the withdrawal end.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within that range, as well as the recited endpoints.

The term “about” as used herein when referring to a measurable value such as a parameter, an amount, and the like, is meant to encompass variations of +/−20% or less, preferably +/−10% or less, more preferably +/−5% or less, even more preferably +/−1% or less, and still more preferably +/−0.1% or less of and from the specified value. Such variations are appropriate to perform the disclosed invention. It is to be understood that the value to which the modifier “about” refers is itself also specifically, and preferably, disclosed.

As used herein, the term “perforations” can also refer to openings, holes, apertures or pores.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

The term “tampon” broadly encompasses any type of absorbent structure used for inserting into openings, orifices, cavities or canals with the purpose of absorbing liquid or moisture potentially present therein or discharged there from. For example, tampons may be configured and intended for inserting into a body cavity or canal of an animal. Preferably the animal is mammal, more preferably human. For example, tampons may be inserted into the vaginal canal, urinal canal, rectum, ear canal, nasal canal or throat. Alternatively, tampons can also be used in such body orifices to deliver substances such as drugs, microflora or moisture. In another example, tampons may be configured to be inserted within industrial installations, equipments or appliances, such as for instance tubing or piping. A preferred tampon type may be a catamenial tampon, configured for insertion into the vaginal canal of human females, for absorbing and preventing the leakage of menstrual fluid.

As detailed above, the present invention concerns advancements to the outer cover of tampons, whereby the overall characteristics and performance of tampons comprising said outer cover are improved. Therefore, the skilled reader shall appreciate that the teachings of the present invention are generally applicable to a wide variety of tampons, including various types and forms of catamenial tampons. The description of tampons, especially of catamenial tampons, serves to exemplify, but not to limit, the types of tampons to which the invention can preferably apply.

The teachings of the invention may be applicable to self-sustaining shape tampons, deformable or fluid-permeable bag tampons. Self-sustaining shape tampons are presently in widespread use, and non-limiting, exemplary disclosures thereof include, e.g., GB 394,571, GB 490,024, DE 3,934,153 C2 and U.S. Pat. No. 5,911,712 incorporated by reference herein. Exemplary deformable, fluid-permeable bag tampons are disclosed in, e.g., U.S. Pat. No. 3,815,601 and U.S. Pat. No. 4,278,088, incorporated by reference herein.

Generally, a tampon may comprise an elongated absorbent core, including an absorbent material, an upper insertion end and a lower withdrawal end. The upper insertion end corresponds to the end which leads the insertion of the tampon into a body cavity. The lower withdrawal end, opposite to the insertion end, guides the withdrawal of the tampon from the body cavity.

According to the invention, a tampon may comprise an elongated absorbent core, an upper insertion end, a lower withdrawal end, and an outer cover having a multiplicity of perforations, wherein the degree of perforation in an upper cylindrical segment is larger than the degree of perforation of a lower cylindrical segment in said outer cover and wherein said upper cylindrical segment is located in the upper part of said outer cover. The absorption and sealing characteristics of the tampon in the body cavity are noticeably increased by the enhanced expansion in the area of the upper insertion end of the tampon. As used herein, the upper part of the outer cover refers to the portion extending from the centre of the outer cover to the upper insertion end of the tampon.

Preferably the outer cover may at least partly ensheathe the elongated absorbent core. For instance, the outer cover may ensheathe at least 50%, preferably at least 75%, more preferably at least 90%, yet more preferably at least 95%, even more preferably at least 98%, or even about 100% of the total area of said elongated absorbent core.

One or both end surfaces of the absorbent core may also be partly or wholly covered by the outer cover. This is usually not necessary to achieve the above-discussed advantages. Hence, in embodiments, either, any one or both end surfaces of the absorbent core are, independently, partly or wholly covered by the outer cover. In a preferred embodiment, the insertion end of the tampon may not be covered by the outer cover. Hereby, the absorbent material at the upper insertion end surface of the absorbent core can instantly contact and absorb fluids when the tampon is inserted into a body cavity.

A preferred tampon of the invention may have a cylindrical shape. The term “cylindrical” does not refer necessarily to a right circular cylinder, but rather to a shape that can be approximated to a geometrical cylinder or a derivative thereof. Hence, the term encompasses a typical cylinder form, a truncated cone form (a frustoconical shape), a barrel form (pineapple shape), oblate or partially flattened cylinder forms, curved cylinder forms, cylindrical forms with varying cross-sectional areas and the like. It shall be appreciated that while a tampon may have an overall cylindrical shape, its surface may include profiles such as variably shaped grooves or ribs. The term “cylindrical” refers to the usual shapes of tampons, especially catamenial tampons, as well-known in the art. Another preferred tampon of the invention may have an enlarged (e.g., preferably mushroom-shaped) head, a barrel shape and/or a conical withdrawal end. The shape is reflected in the longitudinal cross-section of the tampon. The enlarged or mushroom head may be, e.g., quasi-spherical, or may be axially prolonged or flattened. The (slightly) enlarged (widened) distal head secures the tampon against proximal displacement (e.g., when used with a tampon applicator). The tampon may further be provided with a conical withdrawal end. The conical shape is one which is preferably truncated from its point. Such conical end guides the tampon during withdrawal, so making withdrawal easier.

The tampon may preferably comprise a withdrawal cord attached to the withdrawal end to facilitate withdrawal of the tampon after use. The withdrawal cord is preferably flexible, hydrophobic and long enough to protrude from the body cavity. The withdrawal cord is also of sufficient tensile strength to resist breaking during removal of the tampon. A withdrawal cord may be, e.g., a single cord, a tape, or a plurality of strings. Exemplary, non-limiting materials may be cotton, rayon, hydrophobic cotton, hydrophobic polyester or a mixture thereof.

In another preferred embodiment, the tampon of the invention may comprise grooves and/or ribs. Preferably, the grooves may be longitudinal, i.e., extending along at least a portion of the length of the tampon. Preferably, the longitudinal grooves may be generally parallel to the central longitudinal axis of the tampon. Preferably, the longitudinal grooves may extend along at least 50%, preferably at least 70%, more preferably at least 90%, or even more preferably about 100% of the axial length of the elongated surface of the tampon.

A groove may extend over various portions of the tampon circumference. For example, a groove may extend over at least about 10°, 20°, 30°, 40°, 50°, 60°, 70°, 80°, 90°, 100°, 120°, 130°, 140° or 150°.

A tampon may comprise a varying number (even or odd) of such grooves and ribs. The number of grooves and ribs may depend on the diameter of the tampon. The number of grooves and ribs may also depend on the type of absorbent material included within the absorbent core of the tampon. Preferably, a tampon may comprise between 3 and 12 grooves and/or ribs, e.g., 3, 4, 5, 6, 7, 8, 9, 10, 11 or 12, more preferably between 6 and 12, and even more preferably at least about 8 grooves or ribs. Preferably, the longitudinal grooves may be spaced at regular intervals.

The elongated absorbent core of the tampon comprises an absorbent material which can be a hydrophilic material. For example, the absorbent material may be rayon, cotton, wood pulp, comminute wood pulp, creped cellulose wadding, tissue wraps and laminates, peat moss, and chemically stiffened, modified, or cross-linked cellulosic fibres; polymeric materials (e.g. polyester fibres, polyolefin fibres, polyurethane foam, absorbent sponges, super-absorbent polymers, absorbent gelling materials), synthetic fibres, wood pulp or any equivalent materials, or mixtures thereof. The elongated absorbent core may further comprise a second absorbent material over at least a portion of its length. The amount of absorbent material may vary at least over a portion of the length of the tampon. The amount of absorbent material may be higher in the upper part of the tampon than in the lower part of the tampon. The upper part of the tampon refers to the portion extending from the centre of the tampon to the insertion end. The amount of absorbent material may be higher in the upper cylindrical segment than in the lower cylindrical segment.

Moreover, the density of the elongated absorbent core may vary over a portion of its length. The density of the elongated absorbent core may be higher in the upper part of the tampon than in the lower part of the tampon.

A tampon of the invention may further include surface markings (e.g. printed and/or embossed markings), colours or chemical indicators. Moreover, a tampon of the invention may also include odour control agents or antibacterial agents.

The invention concerns improvements to the outer cover of tampons, whereby the overall performances of tampons comprising said outer cover are improved.

The outer cover of the present invention may have a degree of perforation in an upper cylindrical segment, located in the upper part of the outer cover, larger than in a lower cylindrical segment. The term “degree of perforation” as used herein refers to the percentage of the open area of the outer cover, the total perforated area, the average perforations size, the maximal perforation size. At least one of these parameters may be larger in the upper cylindrical segment than in a lower cylindrical segment. Alternatively, more than one or all these parameters may be larger in the upper cylindrical segment than in a lower cylindrical segment. The degree of perforation may vary by increasing the total perforated area or the percentage of the open area, defined by the perforations. Alternatively, the degree of perforation may vary by increasing the average perforations size. Alternatively, the degree of perforation may vary by increasing the maximal perforations size.

The term “cylindrical segment” as used herein refers to a segment taken at right angles to the longitudinal axis Z of a tampon. The cylindrical segment has a diameter approximatively equal to the width of the tampon. The cylindrical segment may be chosen on the tampon surface, but sufficiently far away from the insertion end to avoid insertion end effects. The lower cylindrical segment may be located in any part of the tampon as long as it is located underneath the upper cylindrical segment. In a preferred embodiment, the lower cylindrical segment may be located in the lower part of the outer cover whereas the upper cylindrical segment may be located in the upper part of the outer cover. As used herein, the lower part of the outer cover refers to the portion extending from the centre of the outer cover to the lower withdrawal end.

It is known that the amount of vaginal fluid can change drastically over the menstrual cycle. The amount can change over time on the same person and can vary between persons compared at a similar stage of the monthly period. Solid state particles in vaginal fluid are known to have a blocking effect on the upper part of the tampon. Moreover, high viscosity fluids (like portions of menstrual fluid containing high protein content) are also known to create blockings as they fill up the surface capillaries, thus stopping the fluid absorption. The perforations can avoid or at least reduce blockings since they are less likely to become permanently obstructed. Usually, the blocking may start at a tampon's insertion end, and then gradually expands in the direction of the withdrawal end. However, depending on the situation (e.g. sleeping position of the body user), the blocking can also start at one or more points on the side surface of the tampon and then spread over the entire surface. It is therefore an advantage of the invention to provide an outer cover wherein the degree of perforation in an upper cylindrical segment, located in the upper part of the tampon, is larger than the degree of perforation of a lower cylindrical segment. Hence, the absorption capability of the tampon is improved.

In another embodiment, said upper and lower cylindrical segments may have the same height. In another embodiment, said upper and lower cylindrical segments may have an upper and a lower surface perpendicular to the longitudinal axis of the tampon.

To enhance the advantages brought about by the outer cover having respective areas or segments with comparably greater and comparably smaller degree of perforation, the upper and lower cylindrical segments preferably span relatively sizeable portions of the outer cover of the tampon.

In another embodiment, the height of the upper cylindrical segment H2 may be at least 20% of the height of the outer cover H1. Preferably the height of the upper cylindrical segment may range from about 20 to about 75% of the height H1 of the outer cover, preferably from about 20 to about 50% of the height H1, and more preferably may be 20, 25, 30, 35, 40, 45 or 50% of the height H1, or a value in the range between any two of the aforementioned values.

In another embodiment, the height of the lower cylindrical segment H3 may be at least 20% of the height H1. Preferably the height of the lower cylindrical segment may range from about 20 to about 75% of the height H1 of the outer cover, preferably from about 20 to about 50% of the height H1, and more preferably may be 20, 25, 30, 35, 40, 45 or 50% of the height H1, or a value in the range between any two of the aforementioned values.

In preferred embodiment, the height H2 and H3 of said upper and lower cylindrical segments may be at least 20% of the height H1 of the outer cover. Preferably the height of said upper and lower cylindrical segments respectively, H2 and H3, may range from about 20% to about 75%, more preferably from about 20% to about 50%, and most preferably may be 20, 25, 30, 35, 40, 45, 50% of the height H1 of the outer cover or a value in the range between any two of the aforementioned values.

For example, the height H2 of said upper cylindrical segment may be about 20% of the height H1 of the outer cover, and the height H3 of the lower cylindrical segment may be about 20% of the height H1 of the outer cover.

In another example, the height H2 of said upper cylindrical segment may be about 25% of the height H1 of the outer cover, and the height H3 of the lower cylindrical segment may be about 25% of the height H1 of the outer cover.

Hence, in an embodiment, the height of the upper cylindrical segment H2 may be at least 25% of the height of the outer cover H1. Preferably the height of the upper cylindrical segment may range from about 25 to about 75% of the height H1 of the outer cover, preferably from about 25 to about 50% of the height H1, and more preferably may be 25, 30, 35, 40, 45 or 50% of the height H1, or a value in the range between any two of the aforementioned values.

In another embodiment, the height of the lower cylindrical segment H3 may be at least 25% of the height H1. Preferably the height of the lower cylindrical segment may range from about 25 to about 75% of the height H1 of the outer cover, preferably from about 25 to about 50% of the height H1, and more preferably may be 25, 30, 35, 40, 45 or 50% of the height H1, or a value in the range between any two of the aforementioned values.

In preferred embodiment, the height H2 and H3 of said upper and lower cylindrical segments may be at least 25% of the height H1 of the outer cover. Preferably the height of said upper and lower cylindrical segments respectively, H2 and H3, may range from about 25% to about 75%, more preferably from about 25% to about 50%, and most preferably may be 25, 30, 35, 40, 45, 50% of the height H1 of the outer cover or a value in the range between any two of the aforementioned values.

In yet another example, the height H2 of said upper cylindrical segment may be about 50% of the height H1 of the outer cover and the height H3 of the lower cylindrical segment may be about 50% of the height H1 of the outer cover.

Further, the height H2 of said upper cylindrical segment may be at least 30%, preferably at least 35%, more preferably at least 40% or even more preferably at least 45% of the height H1 of the outer cover and the height H3 of the lower cylindrical segment may be at least 30%, preferably at least 35%, more preferably at least 40% or even more preferably at least 45% of the height H1 of the outer cover.

Also, for example, the height H2 of said upper cylindrical segment may be about 30%, preferably about 35%, more preferably about 40% or even more preferably about 45% of the height H1 of the outer cover and the height H3 of the lower cylindrical segment may be about 30%, preferably about 35%, more preferably about 40% or even more preferably about 45% of the height H1 of the outer cover.

In further embodiments, the outer cover of the tampon may comprise (or define) a number (for example 2 to 10, preferably 3 to 6, such as for example 4 or 5) of notional cylindrical segments of substantially equal height, sequentially arranged along the longitudinal axis of the tampon, wherein the cylindrical segment most proximal to the insertion end of the tampon defines the upper cylindrical segment as intended herein having a greater degree of perforation than any one, preferably more than one and more preferably all, of the remaining cylindrical segments, i.e., the cylindrical segments more distal from the insertion end, which define the lower cylindrical segment(s) as intended herein.

Preferably in these embodiments, the notional cylindrical segments are arranged non-overlappingly and adjoin one another along the longitudinal axis of the tampon, and further preferably the combined height of said cylindrical segments substantially equals the total height of the outer cover.

Hence, in an example the outer cover of the tampon may define 2 sequentially arranged, non-overlapping notional cylindrical segments, each of said segments having height equal to ½ of the total height H1 of the outer cover, wherein the cylindrical segment more proximal to the insertion end of the tampon (i.e., upper cylindrical segment as intended herein) has a greater degree of perforation than the other segment, i.e., the segment more distal from the insertion end (i.e., lower cylindrical segment as intended herein).

In another example the outer cover of the tampon may define 3 sequentially arranged, non-overlapping notional cylindrical segments, each of said segments having height equal to ⅓ of the total height H1 of the outer cover, wherein the cylindrical segment most proximal to the insertion end of the tampon (i.e., upper cylindrical segment as intended herein) has a greater degree of perforation than any one or both of the remaining segments, i.e., the segments more distal from the insertion end (i.e., lower cylindrical segment(s) as intended herein). For instance, when the segments are denoted Sa, Sb and Sc starting from the segment most proximal to the insertion end (Sa) towards the segment most distal from the insertion end (Sc), then the degree of perforation of said segments may be, without limitation, as follows: Sa>Sb≈Sc or Sa>Sb=Sc or Sa>Sb>Sc.

In another example the outer cover of the tampon may define 4 sequentially arranged, non-overlapping notional cylindrical segments, each of said segments having height equal to ¼ of the total height H1 of the outer cover, wherein the cylindrical segment most proximal to the insertion end of the tampon (i.e., upper cylindrical segment as intended herein) has a greater degree of perforation than any one, any two or all three of the remaining segments, i.e., the segments more distal from the insertion end (i.e., lower cylindrical segment(s) as intended herein). For instance, when the segments are denoted Sa, Sb, Sc and Sd starting from the segment most proximal to the insertion end (Sa) towards the segment most distal from the insertion end (Sd), then the degree of perforation of said segments may be, without limitation, as follows: Sa>Sb≈Sc≈Sd or Sa>Sb=Sc=Sd or Sa>Sb>Sc>Sd.

In yet another example the outer cover of the tampon may define 5 sequentially arranged, non-overlapping notional cylindrical segments, each of said segments having height equal to ⅕ of the total height H1 of the outer cover, wherein the cylindrical segment most proximal to the insertion end of the tampon (i.e., upper cylindrical segment as intended herein) has a greater degree of perforation than any one, any two, any three or all four of the remaining segments, i.e., the segments more distal from the insertion end (i.e., lower cylindrical segment(s) as intended herein). For instance, when the segments are denoted Sa, Sb, Sc, Sd and Se starting from the segment most proximal to the insertion end (Sa) towards the segment most distal from the insertion end (Se), then the degree of perforation of said segments may be, without limitation, as follows: Sa>Sb≈Sc≈Sd≈Se or Sa>Sb=Sc=Sd=Se or Sa>Sb>Sc>Sd>Se.

In another preferred embodiment, the height of said upper and lower cylindrical segments, respectively H2 and H3, may be approximatively 50% of the height H1 of said tampon and the degree of perforation in the upper cylindrical segments may be larger than the degree of perforation of said lower cylindrical segments.

In another preferred embodiment, said degree of perforation may be at least 10% larger in the upper cylindrical segment than in the lower cylindrical segment. In a preferred embodiment, the degree of perforation may be from 10 to 500% larger in said upper cylindrical segment than in said lower cylindrical segment, more preferably from 50 to 200% larger in said upper cylindrical segment than in said lower cylindrical segment. Preferably, the degree of perforation may be 50, 75, 100, 125, 150, 175 or 200% larger in said upper cylindrical segment than in said lower cylindrical segment, or a value in the range between any two of the aforementioned values. These values are considered prior to the use of the tampon and may change after the use of the tampon.

In another embodiment, the total perforation area, defined by the perforations, in the upper cylindrical segment may range from about 0.01 mm² to about 10 cm², preferably from about 0.01 mm² to about 5 cm² and more preferably from about 0.1 mm² to about 100 mm². In another embodiment, the total perforation area in the lower cylindrical segment may range from about 0 mm² to about 10 cm², preferably from about 0 mm² to about 5 cm² and more preferably from about 0 mm² to about 100 mm².

In another embodiment, after use of the tampon, the upper surfaces of said upper and lower cylindrical segments have enlarged width compared to their respective lower surfaces in said upper and lower cylindrical segments. Moreover, after use of the tampon, the average width of the upper cylindrical segment may be larger than the average width of the lower cylindrical segment. Due to this arrangement, the withdrawal of the tampon can be facilitated for the user without any fibre loss and the absorption capacity of the tampon is improved.

The perforations of said outer cover may have an inner or an outer diameter. The term “inner diameter” as used herein refers to the diameter of the largest circle that can be inscribed in the perforation. The term “outer diameter” as used herein refers to the diameter of the largest circle that can circumscribe in the perforation. In another embodiment, the average inner or outer diameter of the perforations may increase from said lower cylindrical segment to said upper cylindrical segment.

The size of the perforations may uniformly decrease from the perforations adjacent to the insertion end of the tampon over the total length of the tampon towards the withdrawal end. This can be achieve by a greater size of said perforations in the direction the insertion end of the tampon and also by a slight reduction of the average distance of the perforations from each other in the same direction. Moreover, by selecting the geometrical configuration of the perforations, it may be possible to vary the size of the perforations whilst the observer would not notice whether the perforations are in fact larger or smaller.

In another embodiment, the ratio between the average inner diameter of the perforations of said upper cylindrical segment and the average inner diameter of the perforations of said lower cylindrical segment may be smaller than 15. In a preferred embodiment, the ratio between the average inner diameter of the perforations of said upper cylindrical segment and the average inner diameter of the perforations of said lower cylindrical segment may be smaller than 8. This ratio between inner diameters of the perforations of both cylindrical segments allows a compromise between reducing blocking and limiting fiber loss. Preferably the ratio may range from 1 to 5. More preferably, the ratio may be 1, 1.5, 2.0, 2.5, 3.0, 3.5, 4.0, 4.5 or 5.0, or a value in the range between any two of the aforementioned values.

In another embodiment, the perforations may have an inner or outer diameter ranging from 0.1 mm to 10 mm, preferably from 0.1 mm to 7 mm. More preferably, the perforations may have an inner or outer diameter ranging from 0.1 mm to 5 mm and even more preferably from 0.8 mm to 4 mm. In a preferred embodiment, the perforations may have an inner or outer diameter of about 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9 or 4.0, or a value in the range between any two of the aforementioned values.

In a preferred embodiment, the percentage of the open area defined by the perforations in the upper cylindrical segment may be at least 1.5%, preferably at least 10% and more preferably at least 25% of the surface area of the outer cover in the upper cylindrical segment. In another preferred embodiment, the percentage of the open area defined by the perforations in the lower cylindrical segment may be at least 0%, preferably at least 5% and more preferably at least 10% of the surface area of the outer cover in the lower cylindrical segment.

In another embodiment, the total perforated area defined by the perforations along the outer cover may be at least 1.5%, preferably 15% and more preferably at least 35% of the surface area of the outer cover. This is required to improve the absorption capacity of the tampon and to avoid blocking due to solid state particles or high viscosity fluids.

In another embodiment of the invention, the distance between two perforations of said outer cover may be at least 0.1 mm. Preferably, the distance may range from 0.1 to 10 mm, more preferably from 0.1 to 5.0 mm. In particular, the distance may be 0.1, 0.5, 1.0, 1.5, 2.0, 2.5, 3.0, 3.5, 4.0, 4.5 or 5.0 mm, or a value in the range between any two of the aforementioned values. As used herein, the distance between two perforations is the distance between the two centres of the largest circle that can be inscribed in or circumscribed in both perforations.

In another embodiment, the degree of perforation may also increase along the elongated absorbent core from the lower cylindrical segment to the upper cylindrical segment by increasing the perforation density, which is defined by the number of perforations per square centimetre. The perforation density can improve the absorption rate of the tampon.

Constraints relating to mechanical strength and/or comfort performance may require designs in which perforations with large inner or outer diameter are combined with perforations with small inner or outer diameter. Indeed, small perforations have the advantage to improve the comfort characteristics of a tampon. Moreover, small perforations do not seriously reduce the mechanical strength of the cover. A tampon comprising an outer cover with small perforations is generally smoother to insert and gives rise to less fibre loss.

It is yet another advantage of the invention to combine large perforations with small perforations. Preferably, the small perforations may have an inner or outer diameter ranging from 0.1 mm to 1 mm. Preferably, the large perforation may have an inner or outer diameter ranging from 1 mm to 10 mm. Such range of size can balance the prevention of blocking versus the mechanical strength, the fiber loss and the smoothness of the tampon.

In another embodiment, the perforations may have an area ranging from 0.01 mm² to 100 mm², preferably from 0.01 mm² to 50 mm², more preferably from 0.1 mm² to 10 mm², even more preferably from 0.25 mm² to 5 mm². The perforations may have an area of about 0.1 mm², 0.2 mm², 0.3 mm², 0.4 mm², 0.5 mm², 0.6 mm², 0.7 mm², 0.8 mm², 0.9 mm², 1.0 mm², 1.2 mm², 1.4 mm², 1.6 mm², 1.8 mm² or 2.0 mm², or a value in the range between any two of the aforementioned values. In another embodiment, the average area of the perforations may increase along the elongated absorbent core from said lower cylindrical segment to said upper cylindrical segment.

In another embodiment, the perforations may have the same shape or different shape within a cylindrical segment. In a preferred embodiment, the perforations may be regularly shaped.

In another embodiment, the perforations may be homogeneously distributed in said upper and lower cylindrical segments. A homogeneous distribution of the perforations may ensure an efficient absorption of the vaginal fluid. Alternatively, the perforations may be randomly distributed in said upper and lower cylindrical segments.

In another preferred embodiment, the perforations may have circular, polygonal, oval or elliptical shape; or may be heart-shaped, tear-shaped or diamond-shaped. Preferably, the perforations shape may be circular or polygonal. The term “polygonal” refers to, but is not limited to, triangle, quadrilateral, pentagon, hexagon, heptagon, octagon, nonagon, decagon, undecagon, dodecagon, tridecagon, tetradecagon, pentadecagon, hexadecagon, heptadecagon, octadecagon, nonadecagon, icosagon. Thus, by selecting different geometrical shape, it is possible to vary the degree of perforation of the outer cover. The configuration of the perforations can vary, as desired by the practitioner. For example, the perforations may have substantially parallel sidewalls or the sidewalls may be tapered.

In a preferred embodiment, the outer cover may have hexagonally shaped perforations. In another preferred embodiment, the perforations of said outer cover may have an inner or outer diameter ranging from 1 mm to 2.5 mm. In another preferred embodiment, the perforations may be hexagonally shaped and may have an inner or outer diameter ranging from 1 mm to 2.5 mm.

The outer cover may be made of a fluid-impervious material. Said fluid-impervious material may be in the form of a resilient three-dimensional web. The web may have first and second surfaces, the first surface exhibiting said multiplicity of perforations therein. Each of the fibre-like elements exhibit a cross-section comprising a base portion in the plane of the first surface and a sidewall portion joined to each edge of the base portion. The sidewall portion may extend in the direction of the second surface of the web. The intersecting sidewall portions interconnect to one another the first and second surfaces of the web. The interconnected sidewall portions terminate substantially concurrently with one another in the plane of the second surface. The sidewall portions joined to each edge of the base portion and extending generally in the direction of the second surface of the web improve a mechanical interaction between said sidewall portion and the absorbent material of the elongated absorbent core. Therefore, the cover is securely positioned at the outer surface of the absorbent core in its non-expanded condition and, particularly, also in its expanded position.

In a preferred embodiment, the fluid-impervious material may be a thermoplastic material. By means of preference but not of limitation the thermoplastic material may be polyethylene, polypropylene, other polyolefins, polyethylene terephthalate, ethylene vinyl acetate, polyesters, polystyrenes, polyamides, polyethers, polyurethanes, copolymers or blends thereof. Preferably, the thermoplastic material may be selected from the group comprising polyethylene e.g. linear low density polyethylene, low density polyethylene, high density polyethylene, high molecular weight polyethylene; or polypropylene, ethylene vinyl acetate or copolymers or blends thereof.

In another preferred embodiment, the fluid-impervious material may be an elastic material. The term “elastic” describes the ability of materials to undergo deformation in at least one direction when a force is applied to them and to resume substantially their original dimensions upon relaxing. The elastic elongation of the outer cover may be about 10%, 20%, 30%, 40% 50%, 100%, 150%, 200%, 250%, 300%, 400% or 500%. The elastic material may be selected from the group comprising polyisoprenes, butadiene-styrene copolymers, styrene block copolymers (e.g., styrene/isoprene/styrene (SIS), styrene/butadiene/styrene (SBS), or styrene/ethylene-butadiene/styrene (SEBS) block copolymers), olefinic elastomers, polyether esters, polyurethanes. In addition, additives can be added to the elastic or thermoplastic material e.g. antioxidants, lubricants, antiblock and antislip agents, plasticizers, nucleating agents, antistatic agents, flame retardants, pigments, dyes, and inorganic or organic fillers.

Alternatively, the outer cover may be made of woven fabric or non-woven fabric wherein the perforations are defined by the void spaces between the fabric fibres. As used herein, the term “non-woven” refers to a class of fabrics comprising fibres or threads assembled into a web, wherein the materials are processed into isotropic fabrics by arranging the fibres more or less randomly. The fibres in non-woven materials are interlaid but not in a regular or identifiable manner. As used herein, the term “woven” refers to a class of fabrics comprising fibres, threads or yarns assembled into a web by interlaying in a regular or identifiable manner. The term refers to any woven material such as a woven textile, web, mesh, screen, etc.

The outer cover may further comprise a fibrous material. The term “fibrous material” generally refers to a composite or a laminate comprising two or more materials including fibres. One advantage of this fibrous material is to increase softness of the outer cover. The fibrous material also avoids vaginal irritation when a tampon is withdrawn.

In another embodiment, the outer cover may be provided with a suitable amount of surfactant or coating. This makes the insertion and the withdrawal of the tampon easier. The outer cover can be perforated by any known processes in the art. Several examples include hot air aperturing, and water jet aperturing. Examples of process are disclosed in U.S. Pat. No. 4,695,422 and U.S. Pat. No. 4,741,877.

The tampon may be made by folding or rolling a strip of absorbent material into an elongated shape known as a “tampon blank” and compressing the tampon blank into the tampon's ultimate shape. As used herein, the term “compressing” or “compression” refers to the process of pressing, squeezing, compacting or otherwise manipulating the size, shape, and/or volume of a material. By means of example, the compression or shaping may be in any or all of the width direction, the radial direction, and the axial direction of the tampon. Conditions and manners of compression of a tampon blank are well-known in the art. A withdrawal cord can also be attached either to the tampon blank before compression or following compression to the resulting tampon. An outer cover may be provided on the tampon blank before compression, such that it is compressed simultaneously with the absorbent material to the final tampon shape. Alternatively, an outer cover may be disposed on the absorbent core following compression.

A tampon according to the invention can be useful for capturing body fluids, e.g. vaginal fluids. Further, the teachings of the invention apply to digital tampons, as well as to tampons placed with an applicator. The term “digital tampon” refers to a tampon which is intended to be inserted into the body cavity, especially vaginal canal, with the user's finger and without the aid of an applicator. When a tampon is intended to be digitally inserted, it may be provided with a finger indent or recess at the withdrawal end of the tampon to aid their insertion. In another embodiment, the tampon is configured for insertion with an applicator. The applicator may be any known in the art, e.g., a telescoping-tube type applicator, including the tube-and-plunger type or the compact type arrangements. The applicator may be plastic, paper, or any other suitable material, e.g., moulded polyethylene.

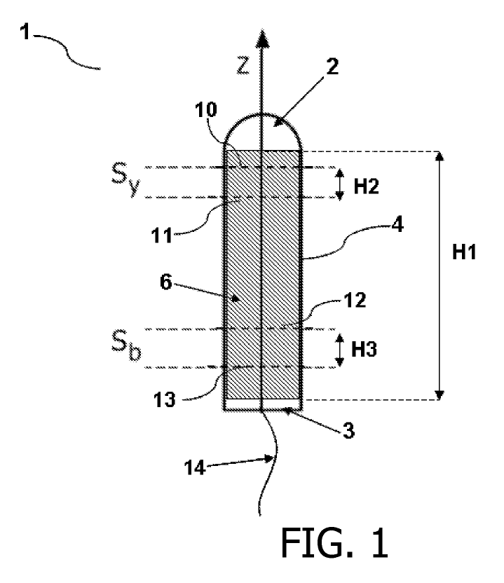

With reference to FIG. 1, a schematic view of a tampon according to an embodiment of the invention is illustrated. A tampon 1 comprises a round dome insertion end 2, a withdrawal end 3, an elongated absorbent core 4 made of fluid absorbing material. The tampon 1 is substantially enclosed within an outer cover 6 having a multiplicity of perforations (not shown). The outer cover 6 is made of a fluid-impervious material in the form of a resilient three-dimensional web. The perforations of the outer cover 6 are different over the length of the tampon 1, so that the tampon 1 will differently expand when applied by body fluid. A withdrawal cord 14 is attached to said tampon 1 and extends from the withdrawal end 3. A Z-axis has been defined along the longitudinal axis of the product. Along this Z-axis, various cylindrical segments S of the tampon can be identified. The height H2 represents the height of the upper cylindrical segment Sy. The height H3 represents the height of the lower cylindrical segment Sb. The height H1 represents the height of the outer cover. The segment Sy has an upper surface 10 and a lower surface 11. The segment Sb has an upper surface 12 and a lower surface 13. The upper and the lower surfaces of both segments are perpendicular to the longitudinal axis Z of the tampon.

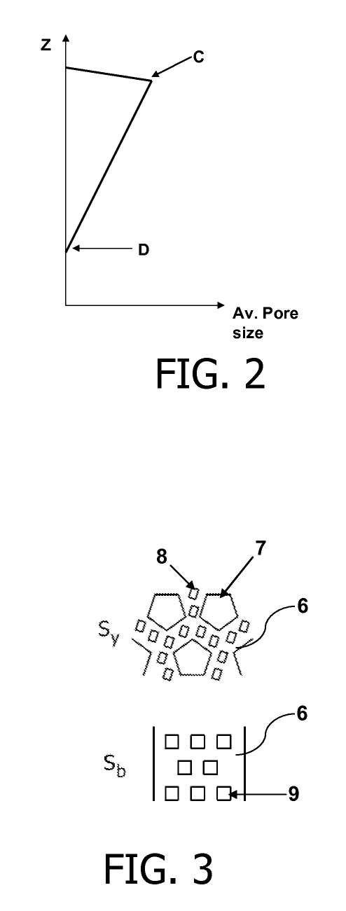

With reference to FIG. 2, a graph of the average pore size distribution along the Z-axis of a tampon according to an embodiment of the invention is illustrated. When making abstraction of the particular effects at the conical tampon insertion end, it has been shown that the average perforation size is largest underneath the insertion end C and gradually decreases towards the withdrawal end D.

With reference to FIG. 3, two segments S of the outer cover according to an embodiment of the invention are illustrated. The segment Sy is located near the insertion end of the tampon and is represented with large perforations 7 and small perforations 8. The perforations 7 are pentagonally shaped. The small perforations 8 are square shaped. The second segment Sb is located near the withdrawal end of the tampon and is represented with large and square-shaped perforations 9. The inner or outer diameter of the perforations 9 is smaller than the inner or outer diameter of the perforations 7 displayed in the upper segment Sy. It will be understood that while segment Sb is represented with only large perforations, said segment can also be represented with a combination of large and small perforations. According to an embodiment of the invention, the degree of perforation of the segment Sy is larger than the degree of perforation of the segment Sb.

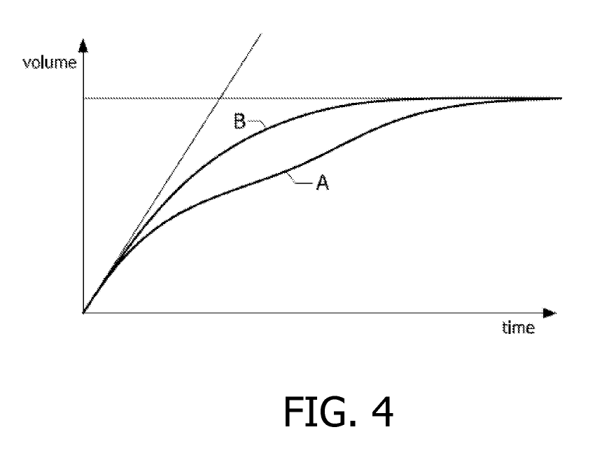

With reference to FIG. 4, a graph of the absorption over time is illustrated. Curve A represents the absorption capacity of a regular size digital tampon with an outer cover in which the perforations are hexagonally shaped and have an inner diameter of 1 mm. Curve B represents the absorption capacity of a regular size digital tampon with an outer cover film in which the perforations are hexagonally shaped and have an inner diameter ranging from 1 mm to 2.5 mm from the withdrawal end to the insertion end of the tampon.

With reference to FIG. 5, a schematic view of a tampon according to an embodiment of the invention is illustrated. The tampon 1 comprises an insertion end 2, a withdrawal end 3, a withdrawal cord 14 and an outer cover 6 having a multiplicity of perforations. The outer cover further comprises two cylindrical segments Sy and Sb wherein the degree of perforation in cylindrical segment Sy is larger than in the cylindrical segment Sb. The cylindrical segment Sy is located in the upper part of said outer cover. Said cylindrical segment Sy comprises a multiplicity of perforations having various shapes e.g. square 51, triangular 52 or round 53. The lower cylindrical segment comprises a multiplicity of perforations 54, 55, 56 having square, triangular or round shape. The outer cover may also comprise perforations 57 which are not located within the cylindrical segment Sb or Sy. These perforations 57 may have any shape allowing efficient absorption of the fluid. The inner or outer diameter of the perforations in the segment Sy is larger than the inner or outer diameter of the perforations in the segment Sb according to an embodiment of the invention. Furthermore, due to this arrangement of the perforations 51 to 56, the absorption capacity of the tampon is enhanced in the direction of the withdrawal end. It will be understood that the perforations 51-56 may have the same shape.

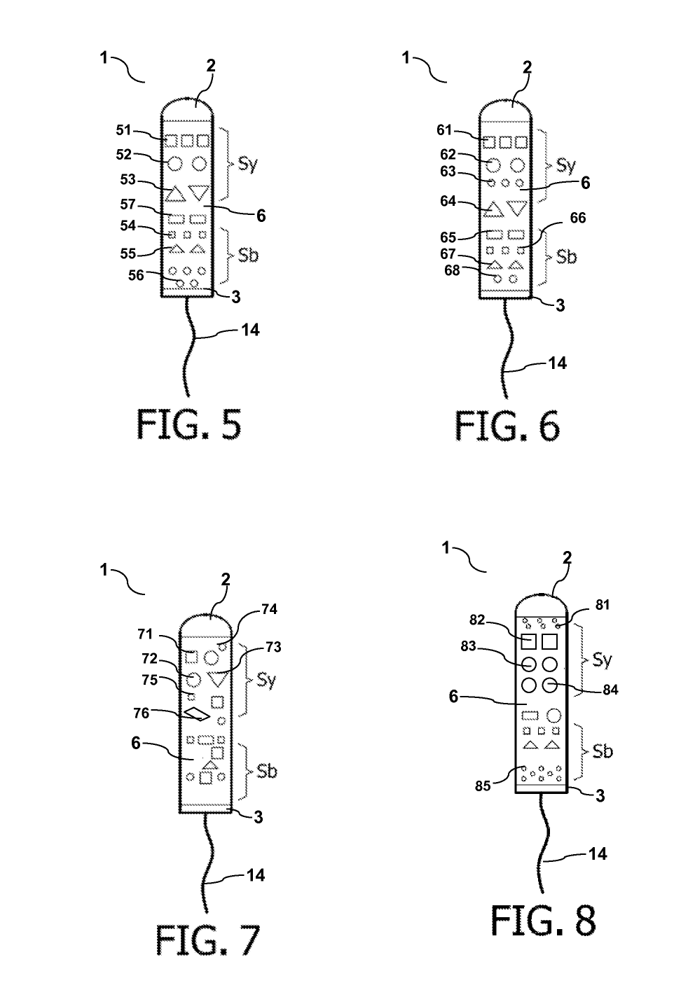

With reference to FIG. 6, a schematic view of a tampon according to an embodiment of the invention is illustrated. The tampon 1 comprises an insertion end 2, a withdrawal end 3, a withdrawal cord 14 and an outer cover 6 having a multiplicity of perforations. The degree of perforation in the segment Sy is larger than the degree of perforation in the segment Sb. The segment Sy comprises a combination of large perforations having different shape (61, 62, and 64) and small perforations 63. It will be understood that the perforations may have the same shape.

With reference to FIG. 7, a schematic view of a tampon according to an embodiment of the invention is illustrated. The tampon 1 comprises an insertion end 2, a withdrawal end 3, a withdrawal cord 14 and an outer cover 6 having a multiplicity of perforations. The degree of perforation in the segment Sy is larger than the degree of perforation in the segment Sb. The segment Sy comprises perforations having random distribution. The segment Sy may comprise a combination of large (71, 72, 73, 76) and small perforations (74, 75). The perforations of the segment Sb can be also randomly distributed.

With reference to FIG. 8, a schematic view of a tampon according to an embodiment of the invention is illustrated. The tampon 1 comprises an insertion end 2, a withdrawal end 3, a withdrawal cord 14 and an outer cover 6 having a multiplicity of perforations. The outer cover has small perforations 81 and 85 proximate to the insertion end 2 and proximate to the withdrawal end 3 respectively. The small perforations 81 proximate to the insertion end 2 have a diameter smaller than the inner or outer diameter of the perforations 82, 83, 84 located in the upper cylindrical segment Sy. Due to the smaller size of the perforations 81 near the insertion end 2 of the tampon 1, the expansion of the insertion end 2 is restricted so that the width of the insertion end of the tampon is reduced.

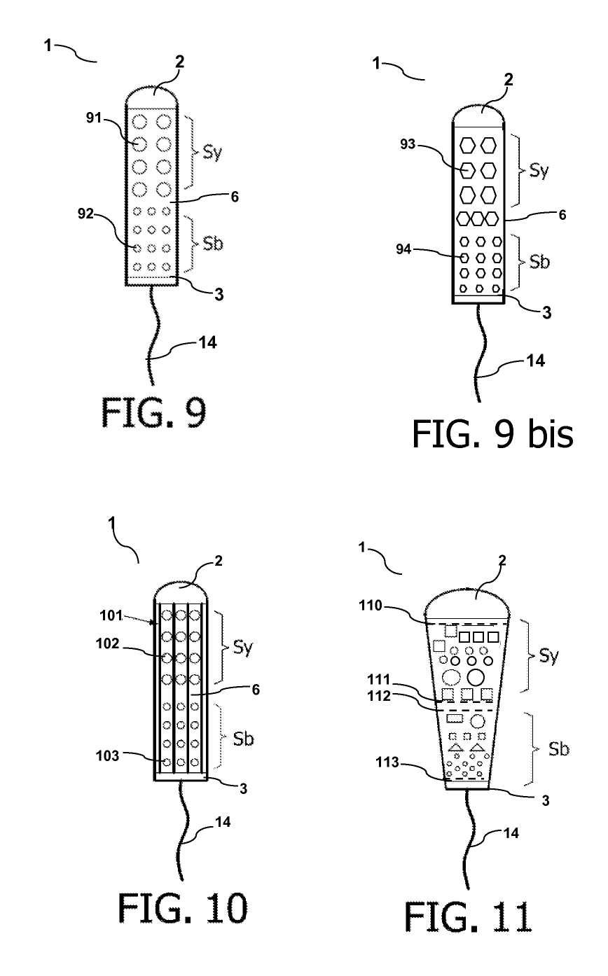

With reference to FIG. 9 and FIG. 9bis, a schematic view of a tampon according to an embodiment of the invention is illustrated. The tampon 1 comprises an insertion end 2, a withdrawal end 3, a withdrawal cord 14 and an outer cover 6 having a multiplicity of perforations. The perforations 91, 92 (FIG. 9) have a round shape and are homogeneously distributed along the elongated absorbent core of the tampon. The perforations 93, 94 (FIG. 9bis) are hexagonally shaped and are homogeneously distributed. The inner diameter of the perforations 93, in the upper segment Sy, is approximatively 2.5 mm whereas the inner diameter of the perforations 94, in the lower segment Sb, is approximatively 1.0 mm. The degree of perforation in segment Sy is larger than the degree of perforation in segment Sb located underneath the segment Sy. The homogeneous distribution ensures an efficient use of the absorption capacity of the tampon.

With reference to FIG. 10, a schematic view of a tampon according to an embodiment of the invention is illustrated. The tampon 1 comprises an insertion end 2, a withdrawal end 3, a withdrawal cord 14 and an outer cover 6 having a multiplicity of perforations. The tampon 1 is further provided with a plurality of grooves 101. The sidewalls of said grooves are covered by said outer cover 6. The perforations 102 and 103 are regularly shaped and homogeneously distributed. However, it will be understood that the perforations can have any shape or can be randomly distributed as long as the degree of perforation in segment Sy is larger than the degree of perforation in segment Sb.

With reference to FIG. 11, a schematic view of a tampon after use according to an embodiment of the invention is illustrated. The tampon 1 comprises an insertion end 2, a withdrawal end 3, a withdrawal cord 14 and an outer cover having a multiplicity of perforations. The upper surfaces 110 and 112 of said upper and lower cylindrical segments have enlarged width compared to their respective lower surfaces 111 and 113 in said upper and lower cylindrical segments. Due to the absorption characteristics controlled by the choice of the size and the position of the perforations, the tampon 1 shows a greater expansion at the insertion end in comparison to the withdrawal end, so that the diameter of the expanded tampon decreases in the direction of its withdrawal end 3. This allows an easier removal of the tampon and avoids friction or fibre loss in the vaginal canal.

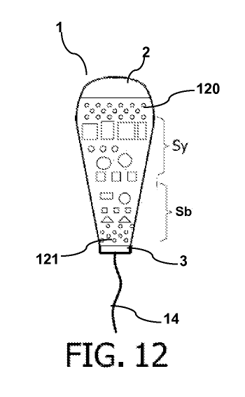

With reference to FIG. 12, a schematic view of a tampon after use according to an embodiment of the invention is illustrated. The tampon 1 comprises an insertion end 2, a withdrawal end 3, a withdrawal cord 14 and an outer cover having a multiplicity of perforations. The outer cover has small perforations 120 and 121 proximate to the insertion end and proximate to the withdrawal end to restrict the width of the tampon proximate to these portions. Due to this arrangement, the absorption capacity of the tampon can be improved and the withdrawal of the tampon can be facilitated.

With regard to the figure, it should be understood that the number of the perforations, the size of the perforations may be increase or decrease depending on the absorption capacity that the manufacturer wants to reach.

It is possible to modify the perforation density, the percentage of open area, the total perforated area, the size of the inner or outer diameter of the perforations, the average area of the perforation in a cylindrical segment, the maximal perforations size and/or the distance between the perforations. It is understandable that it may be possible to combine two, three or all the aforementioned modifications.

An experimental example tests tampons having various heights H2 of an upper cylindrical segment and heights H3 of a lower cylindrical segment compared to the overall height H1 of the outer cover, and tampons with an outer cover defining 3 (Sa, Sb, Sc) or 4 (Sa, Sb, Sc, Sd) notional segments of equal height as described elsewhere in this specification. The tampons are assessed for their ability to absorb an experimental fluid of higher viscosity or an experimental fluid containing particulate matter. The subjective feeling of smoothness upon during insertion or withdrawal of the tampons is recorded in a panel of female users. The results are set out semi-quantitatively in tables below; ‘+’ indicates the presence and extent of an effect.

| H2 (% of H1) | H3 (% of H1) | Absorption | Smoothness |
|---|---|---|---|
| 5% | 5% | − | − |
| 25% | 25% | + | + |
| 30% | 30% | + | + |
| 40% | 40% | ++ | ++ |
| 50% | 50% | +++ | +++ |

| | | |
|---|---|---|
| Degree | | |
| of perforation | Absorption | Smoothness |

3 sequentially arranged, non-overlapping notional
cylindrical segments, each having height equal to ⅓
of the total height H1 (Sa, Sb, Sc)

| | | |
|---|---|---|
| Sa > Sb = Sc | ++ | ++ |
| Sa > Sb > Sc | ++ | ++ |

4 sequentially arranged, non-overlapping notional
cylindrical segments, each having height equal to ¼
of the total height H1 (Sa, Sb, Sc, Sd)

| | | |
|---|---|---|
| Sa > Sb = Sc = Sd | + | + |
| Sa > Sb > Sc > Sd | ++ | ++ |

1. A tampon, comprising an elongated absorbent core, an upper insertion end, a lower withdrawal end, and an outer cover covering the elongated absorbent core and having a multiplicity of perforations,
wherein the degree of perforation in an upper cylindrical segment of said outer cover is larger than the degree of perforation of a lower cylindrical segment of said outer cover and wherein said upper cylindrical segment is located in the upper part of said outer cover and wherein the height of said upper cylindrical segment is at least 20% of the height of the outer cover and the height of the lower cylindrical segment is at least 20% of the height of the outer cover.

2. The tampon according to claim 1, wherein the height of said upper cylindrical segment is at least or about 25% of the height of the outer cover and the height of the lower cylindrical segment is at least or about 25% of the height of the outer cover.

3. The tampon according to claim 1, wherein the height of said upper cylindrical segment is about 50% of the height of the outer cover and the height of the lower cylindrical segment is about 50% of the height of the outer cover.

4. The tampon according to claim 1, wherein the outer cover comprises a number of notional cylindrical segments of substantially equal height, sequentially arranged along the longitudinal axis of the tampon, wherein the upper cylindrical segment most proximal to the insertion end of the tampon has a greater degree of perforation than at least one of the remaining, lower cylindrical segments.

5. The tampon according to claim 4, wherein the outer cover of the tampon comprises 2 sequentially arranged, non-overlapping notional cylindrical segments, each of said segments having height equal to ½ of the total height of the outer cover, wherein the upper cylindrical segment more proximal to the insertion end of the tampon has a greater degree of perforation than the other, lower segment.

6. The tampon according to claim 4, wherein the outer cover of the tampon comprises 3 sequentially arranged, non-overlapping notional cylindrical segments, each of said segments having height equal to ⅓ of the total height of the outer cover, wherein the upper cylindrical segment most proximal to the insertion end of the tampon has a greater degree of perforation than any one or both of the remaining, lower segments.

7. The tampon according to claim 4, wherein the outer cover of the tampon comprises 4 sequentially arranged, non-overlapping notional cylindrical segments, each of said segments having height equal to ¼ of the total height of the outer cover, wherein the upper cylindrical segment most proximal to the insertion end of the tampon has a greater degree of perforation than any one, any two or all three of the remaining, lower segments.

8. The tampon according to claim 4, wherein the outer cover of the tampon comprises 5 sequentially arranged, non-overlapping notional cylindrical segments, each of said segments having height equal to ⅕ of the total height of the outer cover, wherein the upper cylindrical segment most proximal to the insertion end of the tampon has a greater degree of perforation than any one, any two, any three or all four of the remaining, lower segments.

9. The tampon according to claim 1, wherein, after use, the upper surfaces of said upper and lower cylindrical segments have enlarged width compared to their respective lower surfaces in said upper and lower cylindrical segments.

10. The tampon according to claim 1, wherein, after use, the average width of the upper cylindrical segment is larger than the average width of the lower cylindrical segment.

11. The tampon according to claim 1, wherein the ratio between the average inner diameter of the perforations of said upper cylindrical segment and the average inner diameter of the perforations of said lower cylindrical segment is smaller than 8.

12. The tampon according to claim 1, wherein the perforations are homogeneously distributed in said upper and lower cylindrical segments.

13. The tampon according to claim 1, wherein the perforations of said outer cover are regularly shaped.

14. The tampon according to claim 1, wherein the amount of absorbent material varies at least over a portion of the tampon.

15. The tampon according to claim 1, wherein the amount of absorbent material is higher in the upper cylindrical segment than in the lower cylindrical segment.
